(19) United States
(12) Patent Application Publication
(10) Pub. No.: US 2025/0232492 A1
(43) Pub. Date: Jul. 17, 2025

(54) DEEP-LEARNING-DRIVEN ACCELERATED MR VESSEL WALL IMAGING

(71) Applicant: UNIVERSITY OF SOUTHERN CALIFORNIA, Los Angeles, CA (US)

(72) Inventors: Zhaoyang Fan, Los Angeles, CA (US); Zhehao Hu, Los Angeles, CA (US)

(73) Assignee: UNIVERSITY OF SOUTHERN CALIFORNIA, Los Angeles, CA (US)

(21) Appl. No.: 18/843,988

(22) PCT Filed: Mar. 23, 2023
(86) PCT No.: PCT/US2023/016074

§ 371 Date: Sep. 4, 2024

Related U.S. Application Data
(60) Provisional application No. 63/322,997, filed on Mar. 23, 2022.

(51) Int. Cl. G06T 11/00; G06T 3/40; G06T 5/60

(52) U.S. Cl. CPC G06T 11/006; G06T 3/40; G06T 5/60; G06T 2207/10088; G06T 2207/20016; G06T 2207/20052; G06T 2207/20081; G06T 2207/20084; G06T 2207/30101; G06T 2210/41; G06T 2211/441

(57) ABSTRACT

A deep neural network-based reconstruction system for accelerated magnetic resonance imaging of vessel walls. The system can comprise a magnetic resonance imaging (MRI) scanner configured to obtain an image of the vessel walls, and a computer having a processor. The processor comprises a first and second subnetwork implemented in a cascade fashion. The first subnetwork comprises a convolutional neural network (CNN) and an output correcting module. The first subnetwork receives the image and transforms the image to a reduced artifact image. The second subnetwork is an identical duplicate of the first network. The second subnetwork boosts an accuracy of the reduced artifact image to generate a visual representation of the vessel walls. A computer display terminal is connected to the processor and is configured to display the visual representation of the vessel walls.

### CROSS REFERENCE TO RELATED APPLICATIONS

This application claims priority to U.S. Provisional Appl. No. 63/322,997, entitled “DEEP-LEARNING-DRIVEN ACCELERATED MR VESSEL WALL IMAGING” and filed on Mar. 23, 2022, which is herein incorporated by this reference in its entirety.

### STATEMENT AS TO FEDERALLY SPONSORED RESEARCH

This invention was made with government support under grant NIH R01 HL 147355 from the National Institutes of Health (NIH). The government has certain rights in this invention.

### TECHNICAL FIELD

This disclosure generally relates to artificial intelligence and relates more specifically to neural networks used in magnetic resonance (MR) imaging.

### BACKGROUND

MR vessel wall imaging (MR-VWI) can be considered a non-invasive imaging modality that can look beyond the lumen and directly visualize and/or characterize arterial wall lesions involved in various intracranial vasculopathies. Past approaches, such as 3D variable-flip-angle turbo spin-echo, often involve a large spatial coverage and a high spatial resolution that create a prohibitively long acquisition times (e.g., 6-12 minutes per scan). This long acquisition time can be exacerbated when pre- and/or post-contrast scans are ordered (e.g., in atherosclerotic plaque imaging), and the MR-VWI protocol can take at least 15 minutes. This lengthy protocol can then cause low throughput, poor patient tolerance, motion-induced image quality degradation, and even complete scan failure.

While strategies have been proposed to speed up intracranial MR-VWI, these strategies are image domain-based and do not incorporate prior information of data acquired in k-space. Therefore, in view of the above, there is a need for an improved AI system for use in MR-VWI imaging.

### BRIEF DESCRIPTION OF THE DRAWINGS

To facilitate further description of the embodiments, the following drawings are provided in which:

FIG. 1 illustrates a front elevational view of a computer system that is suitable for implementing various embodiments of the systems disclosed in FIGS. 3 and 5;

FIG. 2 illustrates a representative block diagram of an example of the elements included in the circuit boards inside a chassis of the computer system of FIG. 1;

FIG. 3 illustrates a representative block diagram of a system, according to an embodiment;

FIG. 4 illustrates a flowchart for a method, according to an embodiment; and

FIG. 5 illustrates a representative block diagram of a system, according to an embodiment.

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together, but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in some embodiments, the time delay can be less than approximately one second, two seconds, five seconds, or ten seconds.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

### DETAILED DESCRIPTION

A number of embodiments can include a deep neural network-based reconstruction system for accelerated magnetic resonance imaging of vessel walls. In some embodiments, the system can comprise a first and second subnetwork implemented in a cascade fashion, wherein the first subnetwork translates a zero-filling reconstructed image to a reduced artifact image, and wherein the second subnetwork boosts an accuracy of the reduced artifact image.

Some embodiments can include a deep neural network-based reconstruction system for accelerated magnetic resonance imaging of vessel walls. The system can comprise a magnetic resonance imaging (MRI) scanner configured to obtain an image of the vessel walls; a computer having a processor, the processor comprising: a first and second subnetwork implemented in a cascade fashion, wherein the first subnetwork receives the image and transforms the image to a reduced artifact image and wherein the second subnetwork boosts an accuracy of the reduced artifact image to generate a visual representation of the vessel walls, wherein the first subnetwork comprises: a convolutional neural network (CNN); and an output correcting module, wherein the second subnetwork is an identical duplicate of the first network; and a computer display terminal connected to the processor and configured to display the visual representation of the vessel walls.

Additional embodiments can include a method. The method can comprise receiving a magnetic resonance imaging (MRI) scan; feeding the MRI scan into a predictive algorithm; and outputting an improved MRI scan from the predictive algorithm.

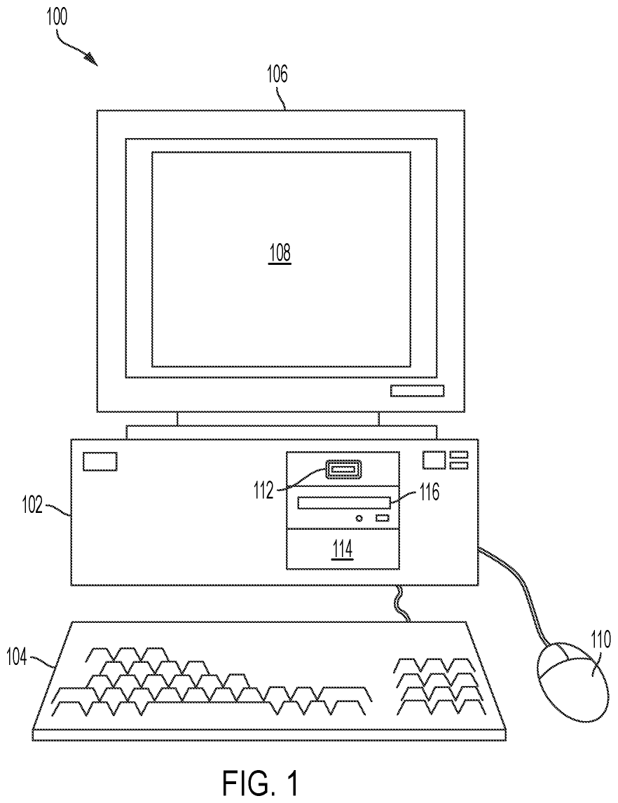

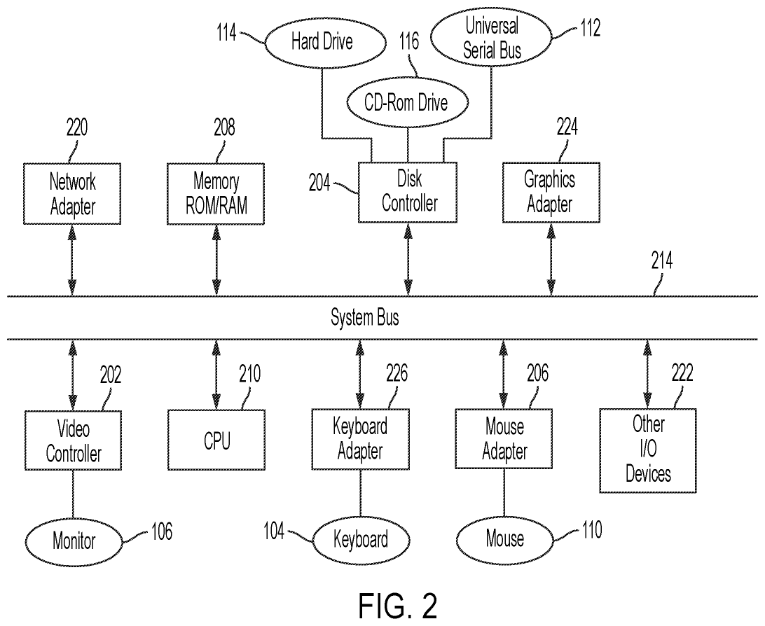

Turning to the drawings, FIG. 1 illustrates an exemplary embodiment of a computer system 100, all of which or a portion of which can be suitable for (i) implementing part or all of one or more embodiments of the techniques, methods, and systems and/or (ii) implementing and/or operating part or all of one or more embodiments of the memory storage modules described herein. As an example, a different or separate one of a chassis 102 (and its internal components) can be suitable for implementing part or all of one or more embodiments of the techniques, methods, and/or systems described herein. Furthermore, one or more elements of computer system 100 (e.g., a monitor 106, a keyboard 104, and/or a mouse 110, etc.) also can be appropriate for implementing part or all of one or more embodiments of the techniques, methods, and/or systems described herein. Computer system 100 can comprise chassis 102 containing one or more circuit boards (not shown), a Universal Serial Bus (USB) port 112, a Compact Disc Read-Only Memory (CD-ROM) and/or Digital Video Disc (DVD) drive 116, and a hard drive 114. A representative block diagram of the elements included on the circuit boards inside chassis 102 is shown in FIG. 2. A central processing unit (CPU) 210 in FIG. 2 is coupled to a system bus 214 in FIG. 2. In various embodiments, the architecture of CPU 210 can be compliant with any of a variety of commercially distributed architecture families.

Continuing with FIG. 2, system bus 214 also is coupled to a memory storage unit 208, where memory storage unit 208 can comprise (i) non-volatile memory, such as, for example, read only memory (ROM) and/or (ii) volatile memory, such as, for example, random access memory (RAM). The non-volatile memory can be removable and/or non-removable non-volatile memory. Meanwhile, RAM can include dynamic RAM (DRAM), static RAM (SRAM), etc. Further, ROM can include mask-programmed ROM, programmable ROM (PROM), one-time programmable ROM (OTP), erasable programmable read-only memory (EPROM), electrically erasable programmable ROM (EEPROM) (e.g., electrically alterable ROM (EAROM) and/or flash memory), etc. In these or other embodiments, memory storage unit 208 can comprise (i) non-transitory memory and/or (ii) transitory memory.

In some embodiments, all or a portion of memory storage unit 208 can be referred to as memory storage module(s) and/or memory storage device(s). In various examples, portions of the memory storage module(s) of the various embodiments disclosed herein (e.g., portions of the non-volatile memory storage module(s)) can be encoded with a boot code sequence suitable for restoring computer system 100 (FIG. 1) to a functional state after a system reset. In addition, portions of the memory storage module(s) of the various embodiments disclosed herein (e.g., portions of the non-volatile memory storage module(s)) can comprise microcode such as a Basic Input-Output System (BIOS) operable with computer system 100 (FIG. 1). In the same or different examples, portions of the memory storage module(s) of the various embodiments disclosed herein (e.g., portions of the non-volatile memory storage module(s)) can comprise an operating system, which can be a software program that manages the hardware and software resources of a computer and/or a computer network. The BIOS can initialize and test components of computer system 100 (FIG. 1) and load the operating system. Meanwhile, the operating system can perform basic tasks such as, for example, controlling and allocating memory, prioritizing the processing of instructions, controlling input and output devices, facilitating networking, and managing files. Exemplary operating systems can comprise one of the following: (i) Microsoft® Windows® operating system (OS) by Microsoft Corp. of Redmond, Washington, United States of America, (ii) Mac® OS X by Apple Inc. of Cupertino, California, United States of America, (iii) UNIX® OS, and (iv) Linux® OS. Further exemplary operating systems can comprise one of the following: (i) the iOS® operating system by Apple Inc. of Cupertino, California, United States of America, (ii) the Blackberry® operating system by Research In Motion (RIM) of Waterloo, Ontario, Canada, (iii) the WebOS operating system by LG Electronics of Seoul, South Korea, (iv) the Android™ operating system developed by Google, of Mountain View, California, United States of America, (v) the Windows Mobile™ operating system by Microsoft Corp. of Redmond, Washington, United States of America, or (vi) the Symbian™ operating system by Accenture PLC of Dublin, Ireland.

As used herein, “processor” and/or “processing module” means any type of computational circuit, such as but not limited to a microprocessor, a microcontroller, a controller, a complex instruction set computing (CISC) microprocessor, a reduced instruction set computing (RISC) microprocessor, a very long instruction word (VLIW) microprocessor, a graphics processor, a digital signal processor, or any other type of processor or processing circuit capable of performing the desired functions. In some examples, the one or more processing modules of the various embodiments disclosed herein can comprise CPU 210.

Alternatively, or in addition to, the systems and procedures described herein can be implemented in hardware, or a combination of hardware, software, and/or firmware. For example, one or more application specific integrated circuits (ASICs) can be programmed to carry out one or more of the systems and procedures described herein. For example, one or more of the programs and/or executable program components described herein can be implemented in one or more ASICs. In some embodiments, an application specific integrated circuit (ASIC) can comprise one or more processors or microprocessors and/or memory blocks or memory storage.

In the depicted embodiment of FIG. 2, various I/O devices such as a disk controller 204, a graphics adapter 224, a video controller 202, a keyboard adapter 226, a mouse adapter 206, a network adapter 220, and other I/O devices 222 can be coupled to system bus 214. Keyboard adapter 226 and mouse adapter 206 are coupled to keyboard 104 (FIGS. 1-2) and mouse 110 (FIGS. 1-2), respectively, of computer system 100 (FIG. 1). While graphics adapter 224 and video controller 202 are indicated as distinct units in FIG. 2, video controller 202 can be integrated into graphics adapter 224, or vice versa in other embodiments. Video controller 202 is suitable for monitor 106 (FIGS. 1-2) to display images on a screen 108 (FIG. 1) of computer system 100 (FIG. 1). Disk controller 204 can control hard drive 114 (FIGS. 1-2), USB port 112 (FIGS. 1-2), and CD-ROM drive 116 (FIGS. 1-2). In other embodiments, distinct units can be used to control each of these devices separately.

Network adapter 220 can be suitable to connect computer system 100 (FIG. 1) to a computer network by wired communication (e.g., a wired network adapter) and/or wireless communication (e.g., a wireless network adapter). In some embodiments, network adapter 220 can be plugged or coupled to an expansion port (not shown) in computer system 100 (FIG. 1). In other embodiments, network adapter 220 can be built into computer system 100 (FIG. 1). For example, network adapter 220 can be built into computer system 100 (FIG. 1) by being integrated into the motherboard chipset (not shown) or implemented via one or more dedicated communication chips (not shown), connected through a PCI (peripheral component interconnector) or a PCI express bus of computer system 100 (FIG. 1) or USB port 112 (FIG. 1).

Returning now to FIG. 1, although some other components of computer system 100 are not shown, such components and their interconnection are well known to those of ordinary skill in the art. Accordingly, further details concerning the construction and composition of computer system 100 and the circuit boards inside chassis 102 are not discussed herein.

Meanwhile, when computer system 100 is running, program instructions (e.g., computer instructions) stored on one or more of the memory storage module(s) of the various embodiments disclosed herein can be executed by CPU 210 (FIG. 2). At least a portion of the program instructions, stored on these devices, can be suitable for carrying out at least part of the techniques and methods described herein.

Further, although computer system 100 is illustrated as a desktop computer in FIG. 1, there can be examples where computer system 100 may take a different form factor while still having functional elements similar to those described for computer system 100. In some embodiments, computer system 100 may comprise a single computer, a single server, or a cluster or collection of computers or servers, or a cloud of computers or servers. Typically, a cluster or collection of servers can be used when the demand on computer system 100 exceeds the reasonable capability of a single server or computer. In certain embodiments, computer system 100 may comprise a portable computer, such as a laptop computer. In certain other embodiments, computer system 100 may comprise a mobile electronic device, such as a smartphone. In certain additional embodiments, computer system 100 may comprise an embedded system.

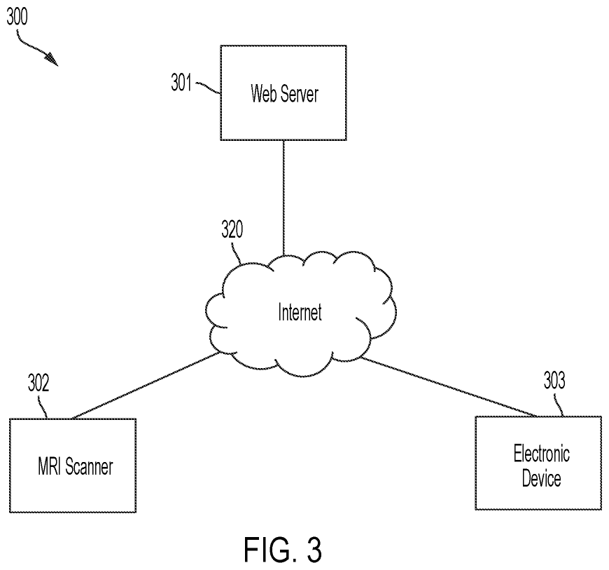

Turning ahead in the drawings, FIG. 3 illustrates a block diagram of a system 300 that can be employed for deep-learning-driven accelerated MR vessel wall imaging, as described in greater detail below. System 300 is merely exemplary and embodiments of the system are not limited to the embodiments presented herein. System 300 can be employed in some different embodiments or examples not specifically depicted or described herein. In some embodiments, certain elements or modules of system 300 can perform various procedures, processes, and/or activities. In these or other embodiments, the procedures, processes, and/or activities can be performed by other suitable elements or modules of system 300.

Generally, therefore, system 300 can be implemented with hardware and/or software, as described herein. In some embodiments, part or all of the hardware and/or software can be conventional, while in these or other embodiments, part or all of the hardware and/or software can be customized (e.g., optimized) for implementing part or all of the functionality of system 300 described herein.

In some embodiments, system 300 can include a web server 301, an MRI scanner 302, and/or an electronic device 303. Web server 301, MRI scanner 302, and/or electronic device 303 can each be and/or incorporate a computer system, such as computer system 100 (FIG. 1), as described above, and can each be a single computer, a single server, or a cluster or collection of computers or servers, or a cloud of computers or servers. In another embodiment, a single computer system can host each of two or more of web server 301, MRI scanner 302, and/or electronic device 303. Additional details regarding web server 301, MRI scanner 302, and/or electronic device 303 are described herein.

Generally speaking, MRI scanner 302 can comprise a machine configured to create a magnetic field and use radio waves to produce internal images of the body (e.g., blood vessels or other biological lumens). In various embodiments, MRI scanner 302 can comprise a magnet configured to cast a magnetic field onto a patient. In some embodiments, a magnetic field can align hydrogen atoms in a patient's body. In some embodiments, MRI scanner 302 can comprise a radio frequency (RF) coil. In further embodiments, a RF coil can be configured to generate RF waves that excite hydrogen atoms in a patient's body. In some embodiments, a RF coil can be configured to receive RF signals emitted from hydrogen atoms after excitation. Received RF signals can then be processed into images displayed on web server 301, MRI scanner 302, and/or electronic device 303 using various mathematical techniques (e.g., by using a Fourier transform). In some embodiments, MRI scanner 302 can comprise shielding (e.g., magnetic shielding) configured to prevent outside signals from interfering with an MRI scan of a patient.

In some embodiments, web server 301, MRI scanner 302, and/or electronic device 303 can be mobile devices. A mobile electronic device can refer to a portable electronic device (e.g., an electronic device easily conveyable by hand by a person of average size) with the capability to present audio and/or visual data (e.g., text, images, videos, music, etc.). For example, a mobile electronic device can comprise at least one of a digital media player, a cellular telephone (e.g., a smartphone), a personal digital assistant, a handheld digital computer device (e.g., a tablet personal computer device), a laptop computer device (e.g., a notebook computer device, a netbook computer device), a wearable user computer device, or another portable computer device with the capability to present audio and/or visual data (e.g., images, videos, music, etc.). Thus, in some examples, a mobile electronic device can comprise a volume and/or weight sufficiently small as to permit the mobile electronic device to be easily conveyable by hand. For examples, in some embodiments, a mobile electronic device can occupy a volume of less than or equal to approximately 1790 cubic centimeters, 2434 cubic centimeters, 2876 cubic centimeters, 4056 cubic centimeters, and/or 5752 cubic centimeters. Further, in these embodiments, a mobile electronic device can weigh less than or equal to 15.6 Newtons, 17.8 Newtons, 22.3 Newtons, 31.2 Newtons, and/or 44.5 Newtons.

Exemplary mobile electronic devices can comprise (i) an iPod®, iPhone®, iTouch®, iPad®, MacBook® or similar product by Apple Inc. of Cupertino, California, United States of America, (ii) a Blackberry® or similar product by Research in Motion (RIM) of Waterloo, Ontario, Canada, (iii) a Lumia® or similar product by the Nokia Corporation of Keilaniemi, Espoo, Finland, and/or (iv) a Galaxy™ or similar product by the Samsung Group of Samsung Town, Seoul, South Korea. Further, in the same or different embodiments, a mobile electronic device can comprise an electronic device configured to implement one or more of (i) the iPhone® operating system by Apple Inc. of Cupertino, California, United States of America, (ii) the Blackberry® operating system by Research In Motion (RIM) of Waterloo, Ontario, Canada, (iii) the Palm® operating system by Palm, Inc. of Sunnyvale, California, United States, (iv) the Android™ operating system developed by the Open Handset Alliance, (v) the Windows Mobile™ operating system by Microsoft Corp. of Redmond, Washington, United States of America, or (vi) the Symbian™ operating system by Nokia Corp. of Keilaniemi, Espoo, Finland.

Further still, the term “wearable user computer device” as used herein can refer to an electronic device with the capability to present audio and/or visual data (e.g., text, images, videos, music, etc.) that is configured to be worn by a user and/or mountable (e.g., fixed) on the user of the wearable user computer device (e.g., sometimes under or over clothing; and/or sometimes integrated with and/or as clothing and/or another accessory, such as, for example, a hat, eyeglasses, a wrist watch, shoes, etc.). In some examples, a wearable user computer device can comprise a mobile electronic device, and vice versa. However, a wearable user computer device does not necessarily comprise a mobile electronic device, and vice versa.

In specific examples, a wearable user computer device can comprise a head mountable wearable user computer device (e.g., one or more head mountable displays, one or more eyeglasses, one or more contact lenses, one or more retinal displays, etc.) or a limb mountable wearable user computer device (e.g., a smart watch). In these examples, a head mountable wearable user computer device can be mountable in close proximity to one or both eyes of a user of the head mountable wearable user computer device and/or vectored in alignment with a field of view of the user.

In more specific examples, a head mountable wearable user computer device can comprise (i) Google Glass™ product or a similar product by Google Inc. of Menlo Park, California, United States of America; (ii) the Eye Tap™ product, the Laser Eye Tap™ product, or a similar product by ePI Lab of Toronto, Ontario, Canada, and/or (iii) the Raptyr™ product, the STAR 1200™ product, the Vuzix Smart Glasses M100™ product, or a similar product by Vuzix Corporation of Rochester, New York, United States of America. In other specific examples, a head mountable wearable user computer device can comprise the Virtual Retinal Display™ product, or similar product by the University of Washington of Seattle, Washington, United States of America. Meanwhile, in further specific examples, a limb mountable wearable user computer device can comprise the iWatch™ product, or similar product by Apple Inc. of Cupertino, California, United States of America, the Galaxy Gear or similar product of Samsung Group of Samsung Town, Seoul, South Korea, the Moto 360 product or similar product of Motorola of Schaumburg, Illinois, United States of America, and/or the Zip™ product, One™ product, Flex™ product, Charge™ product, Surge™ product, or similar product by Fitbit Inc. of San Francisco, California, United States of America.

A mobile MRI scanner can refer to an MRI scanner that can be moved and/or is mounted on wheels. In various embodiments, a mobile MRI scanner can be mounted on a trailer and/or towed by an automobile. In these or other embodiments, an MRI scanner can be brought to a patient's bedside on a cart. Exemplary mobile MRI scanners include the Hyperfine Swoop™ and/or various non-mobile MRI scanners mounted on wheels.

In some embodiments, system 300 can comprise a graphical user interface (“GUI”). In the same or different embodiments, all or a part of a GUI can be part of and/or displayed by one or more of web server 301, MRI scanner 302, and/or electronic device 303. In some embodiments, a GUI can comprise text and/or graphics (image) based user interfaces. In the same or different embodiments, a GUI can comprise a heads up display (“HUD”). When a GUI comprises a HUD, a GUI can be projected onto a medium (e.g., glass, plastic, etc.), displayed in midair as a hologram, or displayed on a display (e.g., monitor 106 (FIG. 1)). In various embodiments, a GUI can be color, black and white, and/or greyscale. In some embodiments, a GUI can comprise a software application running on a computer system, such as computer system 100 (FIG. 1), web server 301, MRI scanner 302, and/or electronic device 303. In the same or different embodiments, a GUI can comprise a website accessed by electronic device 303 through internet 320. In some embodiments, a GUI can comprise a rotatable and/or navigable display of a vessel or some other interior region and/or organ in a patient's body. In the same or different embodiments, a GUI can be displayed as or on a virtual reality (VR) and/or augmented reality (AR) system or display (e.g., a head mounted electronic device and/or an AR scene displayed on a computer system). In some embodiments, an interaction with a GUI can comprise a click, a look, a selection, a grab, a view, a purchase, a bid, a swipe, a pinch, a reverse pinch, etc.

In some embodiments, web server 301, MRI scanner 302, and/or electronic device 303 can each comprise one or more input devices (e.g., one or more keyboards, one or more keypads, one or more pointing devices such as a computer mouse or computer mice, one or more touchscreen displays, a microphone, etc.), and/or can each comprise one or more display devices (e.g., one or more monitors, one or more touch screen displays, projectors, etc.). In these or other embodiments, one or more of the input device(s) can be similar or identical to keyboard 104 (FIG. 1) and/or a mouse 110 (FIG. 1). Further, one or more of the display device(s) can be similar or identical to monitor 106 (FIG. 1) and/or screen 108 (FIG. 1). The input device(s) and the display device(s) can be coupled to the processing module(s) and/or the memory storage module(s) of web server 301, MRI scanner 302, and/or electronic device 303 in a wired manner and/or a wireless manner, and the coupling can be direct and/or indirect, as well as locally and/or remotely. As an example of an indirect manner (which may or may not also be a remote manner), a keyboard-video-mouse (KVM) switch can be used to couple the input device(s) and the display device(s) to the processing module(s) and/or the memory storage module(s). In some embodiments, the KVM switch also can be part of web server 301, MRI scanner 302, and/or electronic device 303. In a similar manner, the processing module(s) and the memory storage module(s) can be local and/or remote to each other.

In some embodiments, web server 301, MRI scanner 302, and/or electronic device 303 can be configured to communicate with each other. In some embodiments, web server 301, MRI scanner 302, and/or electronic device 303 can communicate or interface (e.g., interact) with each other through a network (e.g., through Internet 330). In some embodiments, Internet 330 can be an intranet that is not open to the public. In further embodiments, Internet 330 can be a mesh network of individual systems. In various embodiments, one or more of web server 301, MRI scanner 302, and/or electronic device 303 can use one or more standard communication protocols to communicate through internet 320. In further embodiments, web server 301 and/or MRI scanner 302 (and/or the software used by such systems) can refer to a back end of system 300 operated by an operator and/or administrator of system 300 and electronic device 303 (and/or the software used by such systems) can refer to a front end of system 300 used by a patient and/or physician. In these or other embodiments, the operator and/or administrator of system 300 can manage system 300, the processing module(s) of system 300, and/or the memory storage module(s) of system 300 using the input device(s) and/or display device(s) of system 300.

Meanwhile, in some embodiments, web server 301, MRI scanner 302, and/or electronic device 303 also can be configured to communicate with one or more databases. The one or more databases can comprise a MRI database that contains information about various types of MRI scans. For example, data describing a k-space for past or current MRI scans can be saved in the one or more databases. In some embodiments, data can be deleted from a database when it becomes older than a maximum age. In some embodiments, a maximum age can be determined by an administrator of system 300. In various embodiments, data collected by MRI scanner 302 in real-time can be streamed to a database for storage.

In some embodiments, one or more databases can be stored on one or more memory storage modules (e.g., non-transitory memory storage module(s)), which can be similar or identical to the one or more memory storage module(s) (e.g., non-transitory memory storage module(s)) described above with respect to computer system 100 (FIG. 1). Also, in some embodiments, for any particular database of the one or more databases, that particular database can be stored on a single memory storage module of the memory storage module(s), and/or the non-transitory memory storage module(s) storing the one or more databases or the contents of that particular database can be spread across multiple ones of the memory storage module(s) and/or non-transitory memory storage module(s) storing the one or more databases, depending on the size of the particular database and/or the storage capacity of the memory storage module(s) and/or non-transitory memory storage module(s). In various embodiments, databases can be stored in a cache (e.g., MegaCache) for immediate retrieval on-demand. The one or more databases can each comprise a structured (e.g., indexed) collection of data and can be managed by any suitable database management systems configured to define, create, query, organize, update, and manage database(s). Exemplary database management systems can include MySQL (Structured Query Language) Database, PostgreSQL Database, Microsoft SQL Server Database, Oracle Database, SAP (Systems, Applications, & Products) Database, IBM DB2 Database, and/or NoSQL Database.

Meanwhile, communication between web server 301, MRI scanner 302, electronic device 303, and/or the one or more databases can be implemented using any suitable manner of wired and/or wireless communication. Accordingly, system 300 can comprise any software and/or hardware components configured to implement the wired and/or wireless communication. Further, the wired and/or wireless communication can be implemented using any one or any combination of wired and/or wireless communication network topologies (e.g., ring, line, tree, bus, mesh, star, daisy chain, hybrid, etc.) and/or protocols (e.g., personal area network (PAN) protocol(s), local area network (LAN) protocol(s), wide area network (WAN) protocol(s), cellular network protocol(s), powerline network protocol(s), etc.). Exemplary PAN protocol(s) can comprise Bluetooth, Zigbee, Wireless Universal Serial Bus (USB), Z-Wave, etc.; exemplary LAN and/or WAN protocol(s) can comprise Institute of Electrical and Electronic Engineers (IEEE) 802.3 (also known as Ethernet), IEEE 802.11 (also known as WiFi), etc.; and exemplary wireless cellular network protocol(s) can comprise Global System for Mobile Communications (GSM), General Packet Radio Service (GPRS), Code Division Multiple Access (CDMA), Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/Time Division Multiple Access (TDMA)), Integrated Digital Enhanced Network (iDEN), Evolved High-Speed Packet Access (HSPA+), Long-Term Evolution (LTE), WiMAX, etc. The specific communication software and/or hardware implemented can depend on the network topologies and/or protocols implemented, and vice versa. In some embodiments, exemplary communication hardware can comprise wired communication hardware including, for example, one or more data buses, such as, for example, universal serial bus(es), one or more networking cables, such as, for example, coaxial cable(s), optical fiber cable(s), and/or twisted pair cable(s), any other suitable data cable, etc. Further exemplary communication hardware can comprise wireless communication hardware including, for example, one or more radio transceivers, one or more infrared transceivers, etc. Additional exemplary communication hardware can comprise one or more networking components (e.g., modulator-demodulator components, gateway components, etc.).

The techniques described herein can provide a practical application and several technological improvements. In some embodiments, the techniques described herein can provide for a faster, more accurate, and/or more precise MRI scanning procedure. The techniques described herein can provide a significant improvement over conventional approaches of producing an MRI scan. In some embodiments, the techniques described herein can beneficially make determinations based on dynamic information that includes past MRI scans and/or MRI scans that have occurred during the same day as a patient's scan. In this way, the techniques described herein can avoid problems with stale and/or outdated machine learned models by continually updating. In a number of embodiments, the techniques described herein can advantageously provide an improve MRI scan by minimizing and/or removing artifacts that are created by the MRI scan and not present in vivo.

In some embodiments, the techniques described herein can be used in a way that cannot be reasonably performed using manual techniques or the human mind. For example, the human mind cannot create MRI images because it does not emit a magnetic field or radio waves strong enough to produce an MRI scan. Further, implementing and/or training a predictive algorithm can use extensive data inputs analyzed at speeds that cannot reasonably be performed in the mind of a human.

In some embodiments, the techniques described herein can allow for complete vessel wall imaging investigation (both pre- and post-contrast scans) in only 6 min, as compared to approximately 15 min in current clinical practice.

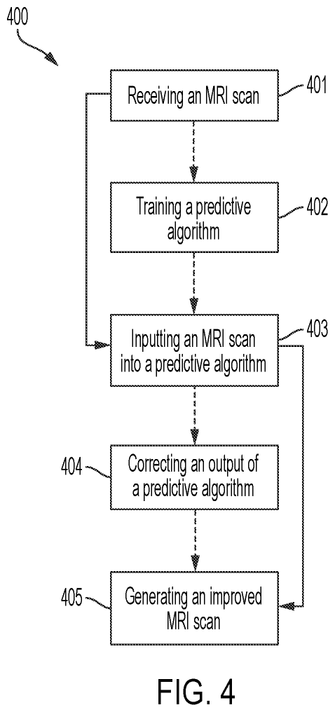

Turning ahead in the drawings, FIG. 4 illustrates a flow chart for a method 400, according to an embodiment. Method 400 is merely exemplary and is not limited to the embodiments presented herein. Method 400 can be employed in some different embodiments or examples not specifically depicted or described herein. In some embodiments, the activities of method 400 can be performed in the order presented. In other embodiments, the activities of method 400 can be performed in any suitable order. In still other embodiments, one or more of the activities of method 400 can be combined or skipped. In some embodiments, system 300 (FIG. 3) can be suitable to perform method 400 and/or one or more of the activities of method 400. In these or other embodiments, one or more of the activities of method 400 can be implemented as one or more computer instructions configured to run at one or more processing modules and configured to be stored at one or more non-transitory memory storage modules. Such non-transitory memory storage modules can be part of a computer system such as web server 301 (FIG. 3), MRI scanner 302 (FIG. 3), and/or electronic device 303 (FIG. 3). The processing module(s) can be similar or identical to the processing module(s) described above with respect to computer system 100 (FIG. 1).

In some embodiments, method 400 can comprise an activity 401 of receiving an MRI scan. In some embodiments, activity 401 can comprise performing a vessel wall imaging protocol that involves an approximately 4-min pre-contrast scan and an approximately 2-min post-contrast scan. In various embodiments, only contrast enhancement information is needed from the post-contrast images if high-quality pre-contrast information is already available due to the similar anatomical structures between the pre- and post-contrast scans.

In various embodiments, an MRI can be received from one or more of web server 301 (FIG. 3) and/or MRI scanner 302 (FIG. 3). For example, an MRI scan can comprise a scan taken of a patient to be enhanced/improved via one or more activities of method 400. As another example, an MRI scan can comprise one or more training MRI scans and/or a training data set used to train a predictive algorithm. Generally speaking, MRI scans received in activity 401 can comprise raw MRI data. In some embodiments, raw MRI data can comprise a raw data matrix. Generally speaking, a raw data matrix can comprise data collected by the MRI machine's receiver coils during an MRI scanning process. In various embodiments, a raw data matrix can comprise a matrix of numbers that represents an amplitude and phase of signals received from a patient at an MRI scanner's (e.g., MRI scanner 302's (FIG. 3)) radio frequency coils. In further embodiments, a raw data matrix can contain a sufficient amount of information needed to reconstruct an image of the patient but cannot be directly interpreted as an image due to its raw formatting. In various embodiments, a raw data matrix can comprise an amplitude, a phase, a frequency, and/or a time entry. In some embodiments, an amplitude can comprise an amplitude of a signal received by an RF coil. In some embodiments, a phase can comprise a phase of hydrogen atoms in the body (e.g., a frequency of spin and/or precess for the hydrogen atoms). In various embodiments, time can comprise a time at which a signal was received by an RF coil. In some embodiments, raw MRI data can comprise data in a k-space. Generally speaking, k-space can comprise a mathematical space generated by MRI scans that contains a processed version of a raw data matrix. In some embodiments, k-space data can be understood as a 2D or 3D grid representing a frequency and phase of radio signals received by an RF coil. A raw data matrix can be converted into a k-space in a number of ways. For example, a Fourier transform can be applied, data can be filtered (e.g., by frequency), a correction can be applied to a phase of the data, magnetic gradients present in the raw data matrix can be encoded to map a signal to a specific location in the patient, and/or a reverse Fourier transform can be applied to transform data into an image domain.

Data received in activity 401 can be stored in a number of different ways. In some embodiments, an MRI scan can be stored in a data store configured to store high dimensional data. For example, high dimensional data can be stored in Facebook AI Similarity Search (AKA “Faiss”) and/or Elastic Search. In the same or different embodiments, high dimensional data can comprise data having a large number of features, thereby leading to “the curse of dimensionality” and therefore slower processing times. In some embodiments, an MRI scan can be stored as a sparse representation in a data store configured to store high dimensional data. Storage efficiency can be improved by encapsulating MRI scans into coarser, conceptual embeddings by storing them as a sparse representation. In some embodiments, a sparse representation of an MRI scan can store only non-zero counts for data entries and/or for vectors created from the data entries. This technique, then, can reduce required storage space, thereby making subsequent reading and/or processing of the sparse representation of an MRI scan faster than reading and/or processing of an MRI scan that is not stored as a sparse representation. In some embodiments, a sparse representation of an MRI scan can be stored in a datastore configured to store high dimensional data, as described above.

In some embodiments, method 400 can optionally comprise an activity 402 of training a predictive algorithm. In other embodiments (e.g., when a pre-trained algorithm is used), activity 402 can be skipped. In some embodiments, training a predictive algorithm can comprise estimating internal parameters of a model configured to remove artifacts from MRI scans. For example, a predictive algorithm can be configured to remove artifacts and/or inaccuracies in an MRI scan caused by undersampling. In various embodiments, a predictive algorithm can be trained using training data, otherwise known as a training dataset. In some embodiments, a training dataset can comprise all or a part of an MRI scan received in activity 401. In this way, a predictive algorithm can be configured to identify and/or remove artifacts in an MRI scan. In the same or different embodiments, a predictive algorithm can comprise a neural network, as described in further detail below.

A pre-trained predictive algorithm can be used, and the pre-trained algorithm can be re-trained on the labeled training data. In some embodiments, a predictive algorithm can also consider both historical and newly added MRI scans when making a prediction. In this way, a predictive algorithm can be trained iteratively as data from MRI scans are added to a training data set. In various embodiments, a predictive algorithm can be trained, at least in part, on a single patient's MRI scans or the single patient's MRI scans can be weighted in a training data set. In this way, a predictive algorithm tailored to a single patient can be generated. In the same or different embodiments, a predictive algorithm tailored to a single patient can be used as a pre-trained algorithm for a similar patient. In several embodiments, due to a large amount of data needed to create and maintain a training data set, a predictive algorithm can use extensive data inputs to enhance and/or improve an MRI scan. Due to these extensive data inputs, in some embodiments, creating, training, and/or using a predictive algorithm configured to enhance and/or improve an MRI scan cannot practically be performed in a mind of a human being.

Training data and/or a training dataset can take a number of forms. For example, training data can be labeled and/or unlabeled. In some embodiments, labeled training data can comprise data in which each data point has an associated label or output value that represents a correct prediction for that data point. For example, in a dataset of MRI scans, each image can be labeled as having artifacts or as not having artifacts. Unlabeled training data can comprise a dataset in which data points do not have associated label or output value. For example, a dataset of MRI scans without labels would be considered unlabeled. Generally speaking, labeled data provides explicit guidance to a predictive algorithm during training while unlabeled data does not provide explicit guidance.

In some embodiments, method 400 can comprise an activity 403 of inputting an MRI scan into a predictive algorithm. In some embodiments, an MRI scan can comprise an MRI scan received in activity 401 and/or an MRI scan collected from a patient. In various embodiments, one or more portions of an MRI scan can be concatenated to form a vector, which can then be inputted into a predictive algorithm. Generally speaking, a predictive algorithm can comprise an algorithm configured to enhance a quality of an MRI scan using probability based algorithms and/or remove deleterious artifacts (e.g., aliasing artifacts) from an MRI scan.

A predictive algorithm can comprise a neural network. Generally speaking, a neural network is a type of machine learning algorithm modeled after the structure and function of the human brain. In various embodiments, a neural network can comprise layers of interconnected neurons. In some embodiments, data input into a neural network can be fed into a first layer and then passed through multiple layers (e.g., hidden layers) of neurons. In some embodiments, an output can be generated at a final layer. In further embodiments, each neuron in a neural network can apply a mathematical operation to data it receives before passing an output for that neuron to a subsequent neuron. In some embodiments, connections between neurons (known as synapses) have a weight that can be adjusted during training. In this way, a performance of the neural network can be optimized. In some embodiments, training a neural network can comprise adjusting weights of synapses so that the network can accurately predict a shape of a bodily structure (e.g., a lumen of a vein).

In some embodiments, a convolutional neural network (CNN) can be used. Generally speaking, a CNN is a type of neural network designed to learn spatial hierarchies of features automatically and adaptively from input data. In some embodiments, a neural network can take undersampled k-space data as inputs and output images with image quality equivalent to those acquired with GRAPPA accelerate of 2×. In some embodiments, a neural network can have a U-Net structure as a backbone CNN architecture. In some embodiments, a CNN can comprise a convolutional layer, a pooling layer, a deconvolutional layer, and/or a fully connected layer. Generally speaking, convolutional layers perform feature extraction using a convolution operation, pooling layers downsample feature maps to reduce a size of data in the network (and thereby increase computational efficiency), deconvolutional layers upsample feature maps to improve further improve the network's ability to learn complex features, and fully connected layers are used to produce an output of the network.

In various embodiments, convolutional layer can implement a type of mathematical operation called a convolution that extracts relevant features from input data. In some embodiments, a convolutional layer can perform convolutions on an MRI scan. In further embodiments, a convolution involves sliding a small matrix (referred to as a kernel or a filter) over input data and performing a dot product between the kernel and the input data. In various embodiments, an output of a convolution can comprise a set of feature maps, each representing a specific feature in the input data. In some embodiments, a feature map can be passed through an activation function (e.g., a ReLU). In further embodiments, an output of an activation function can be passed to a next node or layer in a CNN.

In some embodiments, a CNN can consist of two similar and/or identical subnetworks implemented in a cascading fashion. In some embodiments, a cascading implementation can comprise a first subnetwork that translates a zero-filling reconstructed image to an artifact-free image and a second network that boosts an accuracy of a previous reconstruction. In various embodiments, each subnetwork can have two modules: a multi-scale wavelet CNN module with iterative refinement (e.g., one that implements a discrete wavelet transform and/or an inverse wave transform) and a data consistency module. In these or other embodiments, subnetworks' forward and backward propagations can be well-defined, resulting in one large network that is trainable in an end-to-end manner. In some embodiments, a CNN can have a 2D UNet structure as a backbone network architecture.

To ensure adequate network performance on fine vessel wall structures, modifications can be made to U-Net in at least two aspects. First, pooling and deconvolution operations, which are adopted in conventional U-Net to enlarge receptive field and alter resolution of feature maps, can be replaced by discrete wavelet transform and inverse wavelet transform, respectively. Second, iterative multi-scale refinement blocks are added to fuse coarse features with fine-grained low-level features, thereby restoring sharp vessel wall boundaries. In addition, a data correction (DC) module can be introduced to address data fidelity in a learning stage of the neural network. In some embodiments, two CNN+DC subnetworks can be concatenated together. In this way, a latter subnetwork can act as an extra step to reduce learning errors of an earlier subnetwork.

While some CNNs use a pooling layer to reduce data throughput in the network, pooling layers can decrease feature resolution, thereby increasing chances of generating artifacts and lowering a fidelity of an MRI scan. In some embodiments, a pooling layer in a CNN can be replaced with a discrete wavelet transform (DWT) layer. Generally speaking, DWT is a process used to analyze signals and data in present in different frequency bands. In some embodiments, DWT can decompose a signal into a one or more wavelet coefficients. These coefficients can then be used for analysis or processing of the signal. DWT can be advantageous for MRI scans because it is a multi-resolution analysis technique that captures both high-frequency and low-frequency information in the processed signal. A DWT layer can proceed in a number of ways. In some embodiments, a signal can be decomposed into two parts: an approximation at a lower resolution and a detail at a higher resolution. In some embodiments, a decomposition under DWT can be created using a wavelet filter. In further embodiments, a wavelet filter can comprise a set of coefficients that is convoluted with a signal. Much like in convolutional layers described above, convoluting a wavelet filter can comprise sliding the wavelet filter across an output from a previous node in the network. In some embodiments, an outputs of a convolution using a wavelet filter can be summed to produce an output for the convolution. In various embodiments, convolutions using a wavelet filter can continue until a desired level of resolution is achieved.

DWT can be used in place of a pooling layer to enhance an MRI scan in a number of ways. For example, DWT can be used for image compression. In these embodiments, high-frequency wavelet coefficients can be quantized and discarded while low-frequency coefficients can be encoded and transmitted. In this way, DWT can generate a significant reduction in a size of the image data while preserving features. As another example, DWT can be used for image denoising. In these embodiments, DWT can be used to remove noise from MRI scans by decomposing the signal into wavelet coefficients, thresholding high-frequency coefficients, and then reconstructing the image from remaining coefficients complying with the threshold. In this way, signals outside of the threshold are removed, thereby resulting in improved image quality and reduced noise. As a further example, DWT can be used for image segmentation. In these embodiments, an MRI scan can be decomposed into wavelet coefficients and high-frequency coefficients can be used to identify edges and other features of notes in the MRI scan. In this way, an MRI scan can be separated into different regions or objects.

While some CNNs use a deconvolutional layer to upsample signals in a CNN, deconvolutional layers can lower a fidelity of an MRI scan by causing high frequency information loss. In some embodiments, a deconvolutional layer in a CNN can be replaced with an inverse wavelet transform (IWT) layer. Generally speaking, IWT is a process for reconstructing a signal from its wavelet coefficients, which can be obtained using a wavelet transform (e.g., DWT). In some embodiments, IWT can be used to obtain an approximation of an original signal, which may have been decomposed into wavelet coefficients at multiple scales. IWT can generally be broken down into two steps that are repeated until an original signal is reconstructed: fine scale reconstruction and/or coarse scale reconstruction. In some embodiments, fine scale reconstruction can comprise using coefficients at a finest scale to reconstruct a signal approximation and detail coefficients at that scale. In various embodiments, fine scale reconstruction can involve convoluting scaling and wavelet functions with wavelet coefficients, and then summing the results. In some embodiments, approximation coefficients from a finer scale reconstruction and detail coefficients from a current scale are used to reconstruct approximation coefficients at a coarser scale. In some embodiments, detail coefficients from a finer scale can be up sampled by inserting zeros between adjacent pairs of coefficients, and then convoluting a resulting sequence with a wavelet function for the coarser scale. Coefficients resulting from the convolution can then be added to upsampled approximation coefficients from a finer scale to obtain approximation coefficients for the coarser scale. Approximation coefficients at a coarsest scale can be used as a reconstructed signal. In some embodiments, quality and accuracy of an IWT procedure can be modulated by choosing different wavelet basis functions, a different number of scales, and different thresholding and quantization methods. In various embodiments, an DWT and/or IWN can be configured to concatenate all four subband images in an MRI (e.g., LL, LH, HL, and HH) into a convolutional block without information loss.

In some embodiments, a CNN can comprise an iterative multi-scale refinement (iMR) technique. Generally speaking, iMR can be seen as a process for improving a quality of an MRI scan by iteratively refining wavelet coefficients at one or more scales. In some embodiments, iMR can proceed by applying a thresholding or quantization function to wavelet coefficients at an initial scale. This thresholding or quantization process can then be repeated at each scale until a desired level of refinement is achieved. In this way, delineations between small features in an MRI can he enhanced and artifacts can be minimized or eliminated.

In some embodiments, method 400 can optionally comprise an activity 404 of correcting an output of a predictive algorithm. In some embodiments, activity 405 can be skipped (e.g., when correction is not needed or not desired). In various embodiments, an output of a predictive algorithm (e.g., a predicted improved MRI scan) can be Fourier transformed back into a k-space data. In some embodiments, this newly generated k-space can be replaced with an original acquisition value if a specific location is sampled. In various embodiments, if a location is not sampled, a predicted value (e.g., one generated by a predictive algorithm) can be used in k-space. In various embodiments, updated and/or replaced k-space signals can be back-transformed into an image domain. Image domains generated in activity 400 can then then fed into a subsequent module (e.g., back into a predictive algorithm) or output as final predictions.

In some embodiments, method 400 can comprise an activity 405 of generating an improved MRI scan. In various embodiments, an improved MRI scan can comprise a more real to life reconstruction of an MRI scan as described in activity 401. For example, an improved MRI scan can have sharper delineations between different layers, organs, and/or structures of a patient's body. As a further example, an improved MRI scan can have smoother lines and/or edges. As another example, an improved MRI scan can have fewer or zero artifacts. In some embodiments, an improved MRI scan can be displayed on a GUI (e.g., a GUI as described in FIG. 3).

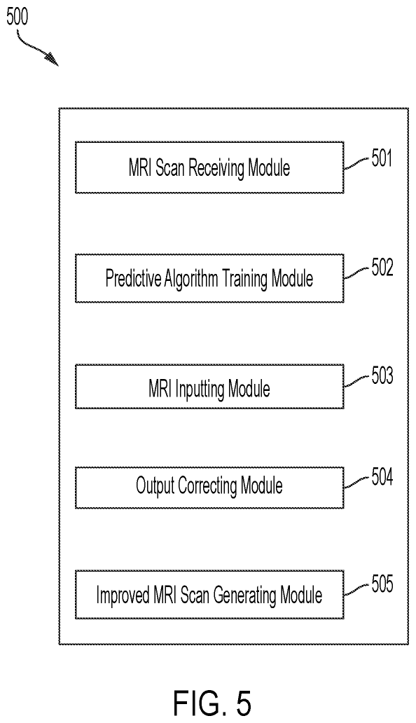

Turning ahead in the drawings, FIG. 5 illustrates a block diagram of a system 500 that can be employed for behavior based messaging. System 500 is merely exemplary and embodiments of the system are not limited to the embodiments presented herein. System 500 can be employed in some different embodiments or examples not specifically depicted or described herein. In some embodiments, certain elements or modules of system 500 can perform various procedures, processes, and/or activities. In these or other embodiments, the procedures, processes, and/or activities can be performed by other suitable elements or modules of system 500. In some embodiments, one or more portions of system 500 can be part of or in communication with web server 301 (FIG. 3), MRI scanner 302 (FIG. 3), and/or electronic device 303 (FIG. 3).

Generally, therefore, system 500 can be implemented with hardware and/or software, as described herein. In some embodiments, part or all of the hardware and/or software can be conventional, while in these or other embodiments, part or all of the hardware and/or software can be customized (e.g., optimized) for implementing part or all of the functionality of system 500 described herein.

In some embodiments, system 500 can comprise non-transitory memory storage module 501. Memory storage module 501 can be referred to as MRI scan receiving module 501. In some embodiments, MRI scan receiving module 501 can store computing instructions configured to run on one or more processing modules and perform one or more acts of method 400 (FIG. 4) (e.g., activity 401 (FIG. 4)).

In some embodiments, system 500 can comprise non-transitory memory storage module 502. Memory storage module 502 can be referred to as predictive algorithm training module 502. In some embodiments, predictive algorithm training module 502 can store computing instructions configured to run on one or more processing modules and perform one or more acts of method 400 (FIG. 4) (e.g., activity 402 (FIG. 4)).

In some embodiments, system 500 can comprise non-transitory memory storage module 503. Memory storage module 503 can be referred to as MRI inputting module 503. In some embodiments, MRI inputting module 503 can store computing instructions configured to run on one or more processing modules and perform one or more acts of method 400 (FIG. 4) (e.g., activity 403 (FIG. 4)).

In some embodiments, system 500 can comprise non-transitory memory storage module 504. Memory storage module 504 can be referred to as output correcting module 504. In some embodiments, output correcting module 504 can store computing instructions configured to run on one or more processing modules and perform one or more acts of method 400 (FIG. 4) (e.g., activity 404 (FIG. 4)).

In some embodiments, system 500 can comprise non-transitory memory storage module 505. Memory storage module 505 can be referred to as improved MRI scan generating module 505. In some embodiments, improved MRI scan generating module 505 can store computing instructions configured to run on one or more processing modules and perform one or more acts of method 400 (FIG. 4) (e.g., activity 405 (FIG. 4)).

Although systems and methods for deep-learning-driven accelerated MR vessel wall imaging have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-5 may be modified, and that the foregoing discussion of certain of these embodiments does not necessarily represent a complete description of all possible embodiments. For example, one or more of the procedures, processes, or activities of FIG. 4 may include different procedures, processes, and/or activities and be performed by some different modules, in some different orders.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. A deep neural network-based reconstruction system for accelerated magnetic resonance imaging of vessel walls comprising:
a first and second subnetwork implemented in a cascade fashion,
wherein the first subnetwork translates a zero-filling reconstructed image to a reduced artifact image, and
wherein the second subnetwork boosts an accuracy of the reduced artifact image.

2. The system of claim 1, wherein the first subnetwork comprises:
a convolutional neural network (CNN); and
an output correcting module.

3. The system of claim 2, wherein the CNN comprises a discrete wavelet transform configured for downsampling and an inverse wavelet transform configured for upsampling.

4. The system of claim 2, wherein the CNN concatenates four subband images of a magnetic resonance imaging scan into a convolutional block.

5. The system of claim 4, wherein the concatenating is without any information loss.

6. The system of claim 5, wherein the CNN further comprises an iterative multi-scale refinement (iMR) block for refining coarse features with fine-grained features at different scales to achieve more accurate wall delineation with sharpened boundaries.

7. The system of claim 2, wherein the output correcting module is after the CNN and configured to enforce data fidelity.

8. The system of claim 7, wherein the output correcting module receives predictions from the CNN as inputs and Fourier transforms the predictions to yield k-space information.

9. The system of claim 8, wherein the output correcting module back-transforms the k-space information to an image domain.

10. The system of claim 9, wherein the back-transformed k-space signals in the image domain are provided to the second subnetwork.

11. The system of claim 10, wherein the second subnetwork is an identical duplicate of the first subnetwork.

12. A deep neural network-based reconstruction system for accelerated magnetic resonance imaging of vessel walls comprising:
a magnetic resonance imaging (MRI) scanner configured to obtain an image of the vessel walls;
a computer having a processor, the processor comprising:
a first and second subnetwork implemented in a cascade fashion, wherein the first subnetwork receives the image and transforms the image to a reduced artifact image and wherein the second subnetwork boosts an accuracy of the reduced artifact image to generate a visual representation of the vessel walls,
wherein the first subnetwork comprises:
a convolutional neural network (CNN); and
an output correcting module,
wherein the second subnetwork is an identical duplicate of the first subnetwork; and
a computer display terminal connected to the processor and configured to display the visual representation of the vessel walls.

13. A method comprising:
receiving a magnetic resonance imaging (MRI) scan;
feeding the MRI scan into a predictive algorithm; and
outputting an improved MRI scan from the predictive algorithm.

14. The method of claim 13, wherein:
the predictive algorithm comprises a neural network having a first and second subnetwork implemented in a cascading fashion;
the first subnetwork removes artifacts from the MRI scan; and
the second subnetwork boosts an accuracy of the first subnetwork.

15. The method of claim 13, wherein:
the MRI scan comprises training data;
the method further comprises:
training the predictive algorithm on the training data, wherein the predictive algorithm is more accurate after the training than before the training; and
the outputting the improved MRI scan comprises:
outputting the improved MRI scan from the trained predictive algorithm.

16. The method of claim 13, wherein the predictive algorithm comprises a convolutional neural network (CNN).

17. The method of claim 13 further comprising:
correcting an output of the predictive algorithm.

18. The method of claim 17, wherein the correcting the output comprises:
correcting the output of the predictive algorithm by applying a Fourier transform to the output of the predictive algorithm.

19. The method of claim 13, wherein a pooling layer in the predictive algorithm is replaced with an inverse wavelet transform layer.

20. The method of claim 13, wherein the improved MRI scan has fewer artifacts and greater resolution than the MRI scan.
